(19) United States
(12) Patent Application Publication
(10) Pub. No.: US 2018/0010175 A1
(43) Pub. Date: Jan. 11, 2018

(54) METHODS AND COMPOSITIONS FOR CLONING CIRCULAR RNA

(71) Applicant: Bio-Rad Laboratories, Inc., Hercules, CA (US)

(72) Inventor: Man CHENG, Danville, CA (US)

(21) Appl. No.: 15/711,215

(22) Filed: Sep. 21, 2017

Related U.S. Application Data
(60) Provisional application No. 62/398,387, filed on Sep. 22, 2016.

(51) Int. Cl. C12Q 1/68; C12P 19/34

(52) U.S. Cl. CPC C12Q 1/6841; C12Q 1/682; C12P 19/34; C12Q 2600/158; C12Q 2545/114

(57) ABSTRACT

Methods and compositions for in situ detection of circular RNA in a tissue sample are provided.

### CROSS-REFERENCE TO RELATED APPLICATION

This application claims priority to U.S. Provisional Application No. 62/398,387, filed Sep. 22, 2016, the contents of which is hereby incorporated by reference in its entirety for all purposes.

### BACKGROUND OF THE INVENTION

A wide variety of circular RNAs have been identified in biological systems. Circular RNAs do not have free 3′ or 5′ ends and instead are covalently linked for form a continuous loop. In some cases, the presence, absence, or quantity (accumulation) of circular RNAs can indicate or be associated with a cellular function or dysfunction (e.g., a disease or disorder). Circular RNAs are described in, e.g., Lasda and Parker, RNA 20:1829-1842 (2014) and Chen, Nature Reviews Molecular Cell Biology 17:205-211 (2016).

### BRIEF SUMMARY OF THE INVENTION

Methods of in situ detection of circular RNA in a tissue or cell sample are described herein. IN some embodiments, the tissue or cell sample is fixed, e.g., formalin fixed. In some embodiments, the method comprises contacting the sample in situ with a strand-displacing RNA polymerase, without initially circularizing nucleic acids in the sample and without contacting circular nucleic acids to the sample, under conditions to allow for rolling circle amplification (RCA) of circular RNA in the sample, if present, to form an RCA amplicon; and detecting the presence, absence or sequence of the RCA amplicon, thereby detecting circular RNA in the tissue sample.

In some embodiments, the strand displacing polymerase is primed by endogenous nucleic acid molecules in the sample.

In some embodiments, the method further comprises adding primer oligonucleotides to the sample and wherein the strand displacing polymerase is primed by primer oligonucleotides. In some embodiments, the primer oligonucleotides are random or degenerate oligonucleotides.

In some embodiments, the strand-displacing RNA polymerase is a reverse transcriptase. In some embodiments, the strand-displacing RNA polymerase is an RNA-dependent RNA polymerase.

In some embodiments, the method further comprises contacting the sample with an exonuclease before the contacting and the detecting, thereby degrading linear nucleic acids.

In some embodiments, the detecting comprises hybridizing a polynucleotide probe to the RCA amplicon. In some embodiments, the hybridizing comprises fluorescence in situ hybridization (FISH). In some embodiments, the polynucleotide probe is linked to a detectable label. In some embodiments, the detectable label comprises a fluorophore.

In some embodiments, the detecting comprises nucleotide sequencing the RCA amplicon. In some embodiments, the detecting comprises determining the sequence of at least one base pair (e.g., a SNP position) by hybridization to the RCA amplicon. In some embodiments, the sequencing occurs in situ.

In some embodiments, the rolling circle amplification introduces multiple synthetic nucleotides comprising moieties available for cross-linking into the RCA amplicon and a cross-linking agent is introduced to cross-link the moieties, thereby forming cross-linked RCA amplicons. In some embodiments, the method further comprises detecting the sequence or one or more contiguous nucleotides in the cross-linked RCA amplicons.

Also provided are mixtures comprising a cell or tissue sample in contact with a strand-displacing RNA polymerase, wherein nucleic acids in the sample have not been circularized by heterologous ligation and wherein the sample has not been contacted with circular nucleic acids.

In some embodiments, the mixture lacks exogenous polynucleotide molecules.

In some embodiments, the mixture further comprises exogenous primer oligonucleotides. In some embodiments, the primer oligonucleotides are random or degenerate oligonucleotides.

In some embodiments, the strand-displacing RNA polymerase is a reverse transcriptase.

In some embodiments, the mixture further comprises an exonuclease that is heterologous to the tissue.

In some embodiments, the mixture further comprises a polynucleotide probe. In some embodiments, the polynucleotide probe is linked to a detectable label. In some embodiments, the detectable label comprises a fluorophore. In some embodiments, the mixture comprises one or more types of synthetic (non-natural) nucleotides comprising a moiety available for cross-linking.

### BRIEF DESCRIPTION OF THE DRAWINGS

FIG. 1 illustrates a possible workflow for generating a rolling circle amplicon from circular RNA in a cell, cross-linking the resulting amplicon, and then detecting the cross-linked amplicons using a downstream detection method. Downstream detection method are described in more detail herein and can include, for example, nucleotide sequencing by synthesis, hybridization of labeled (e.g., fluorescent) oligonucleotide probes, or other methods.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Spring Harbor Lab Press (Cold Spring Harbor, N.Y. 1989). The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention.

“Exogenous” as used herein refers to a molecule that has been added to a sample or tissue, and thus that does not occur naturally. The exogenous molecule can be identical to one that occurs naturally in the sample or tissue (for example the concentration of a natural molecule can be increased by adding more of the molecule to the sample or tissue) or can be different from any molecule that occurs in the sample or tissue.

The terms “label” and “detectable label” interchangeably refer to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELBA), biotin, digoxigenin, ³²P and other isotopes, haptens, proteins, nucleic acids, or other substances which may be made detectable, e.g., by incorporating a label into an oligonucleotide, peptide, or antibody specifically reactive with a target molecule. The term includes combinations of single labeling agents, e.g., a combination of fluorophores that provides a unique detectable signature, e.g., at a particular wavelength or combination of wavelengths.

A molecule that is “linked” to a label (e.g., as for a labeled antibody as described herein) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

### DETAILED DESCRIPTION OF THE INVENTION

### Introduction

The inventor has discovered a method for detecting circular RNA molecules in situ. The method involves treating a tissue sample with a strand-displacing RNA polymerase under conditions such that endogenous circular RNA molecules in the sample are amplified by rolling circle amplification. Rolling circle products can then be detected. For example, probes specific for the products can be used to detect the rolling circle products in situ. Accordingly, methods for detecting circular RNA in a sample in situ, as well as reaction mixtures used in the detection, are provided herein.

### Samples

Any sample containing one or more cells can be used in the methods described herein. The methods can be applied for example to single cells, cell suspensions, or tissue samples. In some embodiments, the sample can be from a eukaryote. Exemplary eukaryotes include, but are not limited to, humans, mice, and plants. In some embodiments, the sample is from an animal, and can be for example, from blood, skin, lung, or other tissue. In some embodiments, the sample is from a healthy individual, or at least from a tissue believed to be healthy. In some embodiments, the sample is from a diseased tissue. An example of a diseased tissue is a sample comprising a tumor. In some embodiments, the sample is a prokaryotic sample. Tissue samples will ideally be thin slices for visualizing signal as well as to allow for penetration of reagents throughout the sample.

Tissue or cell samples will generally be fixed, e.g., formalin fixed or otherwise fixed, thereby terminating ongoing biochemical reactions, preserve tissue/cells and RNA molecules by inactivating protease/nuclease activities.

In general, the sample can be handled under RNase-free conditions to avoid degradation of RNA in the sample. For example, reagents can be treated with DEPC to inhibit any RNase present in the reagents.

In some embodiments, the sample can be permeabilized during or before contact with reagents (e.g., polymerase) intended to enter cells in the sample to allow for better and more efficient penetration of the reagents to cells in the sample. This can be especially effective for tissue samples. In some embodiments, the tissue can be permeabilized by treatment with a protease. For example, pepsin can be used (e.g., at 0.001%-0.5%) to permeabilize the sample. In other embodiments, the cells are permeabilized by contacting the cells with a detergent (e.g., Triton-X100 or SDS), for example at 0.01%-0.5%, or an alcohol (e.g., ethanol), for example at 40-95%, or both.

In many embodiments of the method, the sample will not have been treated to generate circular polynucleotides prior to introduction of the RNA polymerase to generate rolling circle products. Thus for example, in some embodiments, the sample has not been contacted with exogenous circular polynucleotides and has not been treated with a ligase that forms circular polynucleotides.

In some embodiments, the sample is contacted with an exonuclease to degrade linear polynucleotides (e.g., linear RNAs) before the sample is contacted with the polymerase. This can be advantageous for example when linear polynucleotides interfere with, or create background signaling when, detecting rolling circle product, for example that might occur when rolling circle products are detected with a probe or otherwise detected. The sample can be incubated with the exonuclease for a sufficient time to penetrate and degrade linear nucleic acids. In some embodiments, the exonuclease degrades linear RNA. In some embodiments, the exonuclease degrades linear DNA. In some embodiments, the exonuclease degrades linear RNA and linear DNA. Exemplary exonucleases include RNase R and Terminator™ 5′-Phosphate-Dependent Exonuclease (e.g., from Epicentre®, Madison, Wis.) or Exonuclease T (New England BioLabs). Optionally the exonuclease can be inactivated prior to generation of the rolling circle product. Exonucleases can be inactivated for example by application of heat, or in some embodiments, by contacting the enzyme with an oligonucleotide lacking 5′ phosphorylation, which blocks activity of some exonucleases.

### Rolling Circle Amplification

Because the methods generate rolling circle amplification products from endogenous circularized RNA in the sample, in some embodiments, the rolling circle products are generated by simply contacting cells in the sample with an RNA polymerase capable of making a rolling circle product. In some embodiments, the polymerase will be primed by polynucleotides (including but not limited to miRNAs or other RNA molecules) that hybridize to the circularized RNA in the sample. In these embodiments, an exonuclease is not generally added previously, or if added, is DNA-specific, leaving RNA available to primer reaction. In other embodiments, one or more (e.g., linear) polynucleotides can be added to the sample to hybridize to the circularized RNA molecules and prime the polymerase reaction. In some of these embodiments, the sample is contacted with an exonuclease to remove endogenous potential priming polynucleotides and then exogenous polynucleotides are added. In other embodiments, an exonuclease is not added prior to addition of exogenous priming polynucleotides (for example when endogenous polynucleotides do not interfere with amplification from the exogenous polynucleotides). In some embodiments, the polynucleotides are oligonucleotide primers (e.g., between 4-100, e.g., 4-50 nt long). In some embodiments, a single polynucleotide (in multiple copies, but one sequence) is applied to the sample. In some embodiments, multiple polynucleotides of different sequence (each in multiple copies) are applied to the sample. In some embodiments, the polynucleotides are targeted to one or more than one circularized RNA sequence. In another example, the polynucleotides are random or degenerate. For example, where the full scope of circularized RNA in the sample is to be detected, in some embodiments, random primers (e.g., random hexamers) can be applied to the sample. The added exogenous polynucleotides can be RNA, DNA, combinations thereof and/or can comprise or be composed of synthetic or modified nucleotides.

Exemplary RNA polymerases include strand displacing RNA polymerases, including but not limited to, reverse transcriptases. Any of a variety of reverse transcriptases can be used. Exemplary reverse transcriptases include but are not limited to murine leukemia virus (MLV) reverse transcriptase, Avian Myeloblastosis Virus (AMV) reverse transcriptase, Respiratory Syncytial Virus (RSV) reverse transcriptase, Equine Infectious Anemia Virus (EIAV) reverse transcriptase, Rous-associated Virus-2 (RAV2) reverse transcriptase, SUPERSCRIPT II reverse transcriptase, SUPERSCRIPT I reverse transcriptase, THERMOSCRIPT reverse transcriptase and MMLV RNase H⁻ reverse transcriptases. In additional embodiments, a DNA polymerase that functions as an RNA polymerase can be used. For example, Tth and Z05, which are DNA polymerases, can function as reverse transcriptase in the presence of manganese. At higher concentrations, some polymerases affect RNA integrity due to intrinsic RNaseH activity, or result in primer self-amplification due DNA toehold-mediated displacement and DNA-dependent DNA polymerase activity of reverse transcriptase. Because of these reasons, in some embodiments, the reverse transcriptase or RNA-dependent RNA polymerase lacks or substantially lacks RNAse H activity.

Other stand displacing RNA polymerases can also be used, including but not limited to, RNA-dependent RNA polymerases. Exemplary RNA-dependent RNA polymerases include, but are not limited to, those from viruses containing positive-strand RNA or double-strand RNA, negative-strand RNA viruses with segmented genomes, e.g., Orthomyxoviruses (including influenza A, B, and C viruses, Thogotoviruses, and the infectious salmon anemia virus), Arenaviruses, Bunyaviruses, Hantaviruses, Nairoviruses, Phleboviruses, Tenuiviruses and Tospoviruses, and the Birnaviridae family of dsRNA viruses. The concentration of the reverse transcriptase or RNA dependent RNA polymerase can vary and optimal concentrations can be determined empirically and depend on the particular reverse transcriptase used. In some embodiments, the concentration of the strand-displacing RNA polymerase is e.g., 1-40 U/μg tissue, e.g., 15-25 U/μg. In some embodiments, the concentration is 1000, 2000, 3000, 4000, 5000, 6000, 7000, 8000, 9000, 10000, 15000, 20000, 25000, 30000, 350000, or 40000 units (per slide), or within a range between any two of the listed valued. The specific concentration will depend in part on the specific activity of the enzyme used.

Conditions for formation of rolling circle products by the RNA polymerase in a tissue sample will depend on the permeability of the tissue as well as the abundance of circular RNA molecules. Thus, to compensate for low permeability, the RNA polymerase can be incubated with the tissue for a longer time. In some embodiments, the RNA polymerase is incubated with the tissue for 1-24 hours, for example from 2-10 hours, ideally under optimal temperature for activity of the RNA polymerase (e.g. 37° C.).

### Detection of Rolling Circle Products

Once the rolling circle products have been generated in situ in the sample from the circularized RNA templates, the resulting rolling circle products can be detected and quantified as desired. For example, the rolling circle products can be detected by hybridizing a nucleic acid probe to the rolling circle product and detecting or quantifying hybridization of the probe. In some embodiments, the nucleotide sequence of the rolling circle product can be determined.

In some embodiments, one or more nucleic acid probe is hybridized under conditions to allow for specific hybridization of a target rolling circle product sequence. Appropriate hybridization can be determined empirically. The nucleic acid probe can be of any length. For specific binding, the nucleic acid probe will be at least 15 nucleotides long, and in some embodiments 18-50 or more nucleotides long. A detectable label can be directly (covalently) linked to the nucleic acid probe or a detectable label can be added label for example in cases where the nucleic acid probe comprises one member of a specific binding pair and the detectable label is linked to a different specific binding pair. Examples of specific binding pairs include, e.g., biotin and streptavidin or an antibody and its target epitope. Any kind of detectable label can be used, including but not limited to fluorescent or chemiluminescent, or other labels. In some embodiments, multiple nucleic acid probes are added that are linked to different detectable labels such that the signal of the different detectable labels can be differentially detected.

In some embodiments, one or more rolling circle product can be amplified. For example, in situ PCR methods have been described. See, e.g., Bagasra, Nat Protoc. 2007; 2(11):2782-95.

In some embodiments, the rolling circle products are detected by fluorescence in situ hybridization (FISH). FISH refers to a nucleic acid hybridization technique that employs a fluorophor-labeled probe to specifically hybridize to and thereby, facilitate visualization of, a target nucleic acid. Such methods are well known to those of ordinary skill in the art and are disclosed, for example, in U.S. Pat. No. 5,225,326; U.S. patent application Ser. No. 07/668,751; PCT WO 94/02646, the entire contents of which are incorporated herein by reference. In general, in situ hybridization is useful for determining the distribution of a nucleic acid in a nucleic acid-containing sample such as is contained in, for example, tissues at the single cell level.

In some embodiments, the rolling circle products are sequenced in situ. In some embodiments, a small number of nucleotides (e.g., 1, 2, 3, or 4) are determined, for example by specific hybridization and detection of nucleic acid probes. For example, nucleic acid probes differing by a single nucleotide can be added to the sample in situ under stringent conditions, and based on which probe binds, a nucleotide of the rolling circle product can be determined.

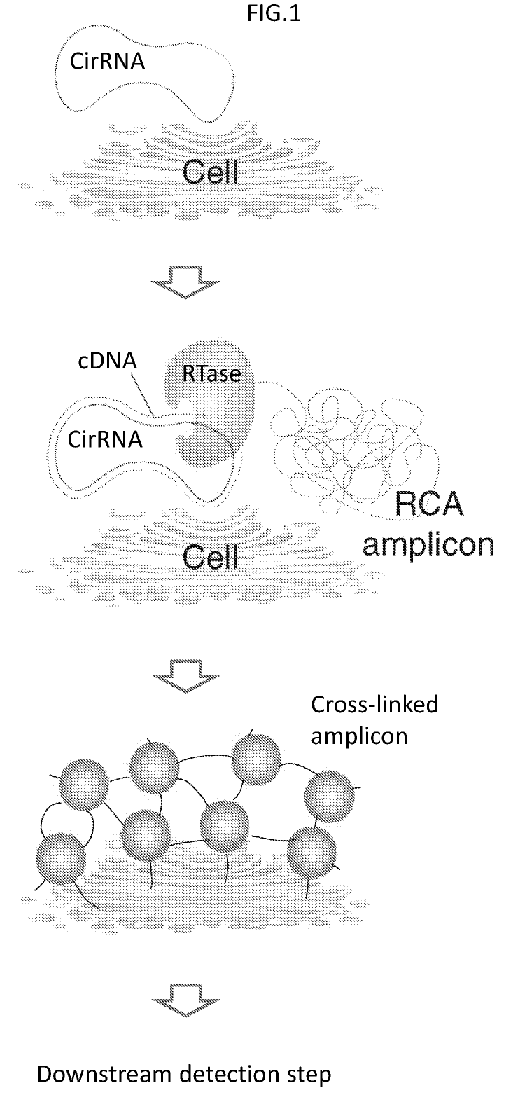

In some embodiments, the sequencing method involves forming rolling circle amplicons in situ that are subsequently cross-linked, thereby mainlining their position in the sample. This can be achieved by performing rolling circle amplification using at least one synthetic nucleotide allowing for a cross-linking moiety that can be used to cross-link the moiety to another moiety incorporated into the same or a different amplicon. Methods for forming a matrix are described in, e.g., WO 2014/163886, which described in part: “the nucleic acids are modified to incorporate a functional moiety for attachment to the matrix. The functional moiety can be covalently cross-linked, copolymerize with or otherwise non-covalently bound to the matrix. The functional moiety can react with a cross-linker. The functional moiety can be part of a ligand-ligand binding pair. dNTP or dUTP can be modified with the functional group, so that the function moiety is introduced into the DNA during amplification. A suitable exemplary functional moiety includes an amine, acrydite, alkyne, biotin, azide, and thiol. In the case of crosslinking, the functional moiety is cross-linked to modified dNTP or dUTP or both. Suitable exemplary cross-linker reactive groups include imidoester (DMP), succinimide ester (NETS), maleimide (Sulfo-SMCC), carbodiimide (DCC, EDC) and phenyl azide. Cross-linkers within the scope of the present disclosure may include a spacer moiety. Such spacer moieties may be functionalized. Such spacer moieties may be chemically stable. Such spacer moieties may be of sufficient length to allow amplification of the nucleic acid bound to the matrix. Suitable exemplary spacer moieties include polyethylene glycol, carbon spacers, photo-cleavable spacers and other spacers known to those of skill in the art and the like.” Such methods used in in situ sequencing are described in, e.g., Lee, et al., Nature Protocols 10(3): 442-458 (2015) and can be adapted to the present method by performing rolling circle amplicon directly from circular RNA in the sample as described herein using at least one synthetic dNTP having a moiety available for cross-linking. Once the cross-linked rolling circle amplicons have been cross-linked, nucleotide sequencing can be performed in situ. These aspects are depicted in FIG. 1.

Methods of sequencing nucleic acid in situ within a matrix can involve general sequencing methods known in the art, such as sequencing by extension with reversible terminators, fluorescent in situ sequencing (FISSEQ), pyrosequencing, massively parallel signature sequencing (MPSS) and the like (described in Shendure et al. (2004) Nat. Rev. 5:335. See, e.g., sequencing methods described in WO 2014/163886. In some embodiments, the nucleic acids within the matrix can be interrogated using methods known to those of skill in the art including fluorescently labeled oligonucleotide/DNA/RNA hybridization, primer extension with labeled ddNTP, sequencing by ligation or sequencing by synthesis.

All patents, patent applications, and other published reference materials cited in this specification are hereby incorporated herein by reference in their entirety.

What is claimed is:

1. A method of in situ detection of circular RNA in a tissue or cell sample, the method comprising,
contacting the sample in situ with a strand-displacing RNA polymerase, without initially circularizing nucleic acids in the sample and without contacting circular nucleic acids to the sample, under conditions to allow for rolling circle amplification (RCA) of circular RNA in the sample, if present, to form an RCA amplicon; and
detecting the presence, absence or sequence of the RCA amplicon, thereby detecting circular RNA in the tissue sample.

2. The method of claim 1, wherein the strand displacing polymerase is primed by endogenous nucleic acid molecules in the sample.

3. The method of claim 1, further comprising add primer oligonucleotides to the sample and wherein the strand displacing polymerase is primed by primer oligonucleotides.

4. The method of claim 3, wherein the primer oligonucleotides are random or degenerate oligonucleotides.

5. The method of claim 1, wherein the strand-displacing RNA polymerase is a reverse transcriptase.

6. The method of claim 1, wherein the strand-displacing RNA polymerase is an RNA-dependent RNA polymerase.

7. The method of claim 1, further comprising contacting the sample with an exonuclease before the contacting and the detecting, thereby degrading linear nucleic acids.

8. The method of claim 1, wherein the detecting comprises hybridizing a polynucleotide probe to the RCA amplicon.

9. The method of claim 1, wherein the hybridizing comprises fluorescence in situ hybridization (FISH).

10. The method of claim 8, wherein the polynucleotide probe is linked to a detectable label.

11. The method of claim 10, wherein the detectable label comprises a fluorophore.

12. The method of claim 1, wherein the detecting comprises nucleotide sequencing the RCA amplicon.

13. The method of claim 1, wherein the rolling circle amplification introduces multiple synthetic nucleotides comprising moieties available for cross-linking into the RCA amplicon and a cross-linking agent is introduced to cross-link the moieties, thereby forming cross-linked RCA amplicons.

14. The method of claim 13, further comprising detecting comprises detecting the sequence or one or more contiguous nucleotides in the cross-linked RCA amplicons.

15. The method of claim 1, wherein the detecting comprises determining the sequence of at least one base pair (e.g., a SNP position) by hybridization to the RCA amplicon.

16. A mixture comprising a cell or tissue sample in contact with a strand-displacing RNA polymerase, wherein nucleic acids in the sample have not been circularized by heterologous ligation and wherein the sample has not been contacted with circular nucleic acids.

17. The mixture of claim 16, wherein the mixture lacks exogenous polynucleotide molecules.

18. The mixture of claim 16, wherein the mixture further comprises exogenous primer oligonucleotides.

19. The mixture of claim 18, wherein the primer oligonucleotides are random or degenerate oligonucleotides.

20. The mixture of claim 16, wherein the strand-displacing RNA polymerase is a reverse transcriptase.

21. The mixture of claim 16, further comprising an exonuclease that is heterologous to the tissue.

22. The mixture of claim 16, further comprising a polynucleotide probe.

23. The mixture of claim 22, wherein the polynucleotide probe is linked to a detectable label.

24. The mixture of claim 23, wherein the detectable label comprises a fluorophore.
